(19) United States
(12) Patent Application Publication
(10) Pub. No.: US 2004/0221855 A1
(43) Pub. Date: Nov. 11, 2004

(54) Methods for monitoring treatment of disease

(76) Inventor: Paul Ashton, Boston, MA (US)

Correspondence Address: ROPES & GRAY LLP, ONE INTERNATIONAL PLACE, BOSTON, MA, 02110-2624, US

(21) Appl. No.: 10/688,151

(22) Filed: Oct. 17, 2003

Related U.S. Application Data
(60) Provisional application No. 60/419,484, filed on Oct. 17, 2002.
(60) Provisional application No. 60/468,964, filed on May 7, 2003.

(51) Int. Cl. A61B 19/00

(52) U.S. Cl. 128/898

(57) ABSTRACT

The invention provides statistical methods for identifying measurements that can be used to generate variables that are associated with clinical outcomes of treatment regimens for disease. The invention also provides methods for using such measurements to monitor the effectiveness of an ongoing disease treatment regimen, databases which contain information about measurements and variables and their relationships to clinical outcomes, and pharmaceutical products which incorporate instructions on the use of the methods and databases of the invention. The invention provides a specific application of these methods and products to the treatment of ocular diseases, particularly macular edema, in particular for the treatment of macular edema with implantable devices and compositions that provide sustained release of corticosteroids.

### FIELD OF THE INVENTION

This invention relates to the fields of statistical analysis, medicine, and pharmaceuticals. More specifically, it relates to methods for determining and monitoring the effects of medical treatments on patients, both in clinical trials and in the provision of medical care. Most specifically, the present invention relates to the diagnosis, monitoring, management and treatment of macular diseases.

### BACKGROUND

The treatment of a subject with a particular disease treatment regimen, whether it be drug administration, surgery, or other form of therapy, will in general have multiple measurable effects on the subject's physiology. For example, levels of various blood components, levels of expression of various genes, and the size and shape of physical features can all be altered by any given treatment regimen. Such changes can be measured today by a wealth of biomedical analytical techniques, which creates the potential for detailed and highly informative monitoring of patients' responses to medical treatment regimens.

It is often not evident, however, which of the multitude of measurable changes are associated with a positive clinical outcome, which are associated with undesirable side-effects, and which are inconsequential. For example, in the treatment of AIDS, measures of HIV viral load and T-cell counts are changes that were expected to be associated with a favorable outcome, and these measures are now accepted as surrogate endpoints in clinical trials of AIDS drugs. However, if a drug being administered to a subject for the treatment of AIDS is found to raise the blood level of a particular interleukin by some measurable amount, it will not be immediately apparent whether this is associated in any way with favorable clinical endpoints such as a reduced infection rate or an extended survival time.

The need to shorten the duration and cost of clinical trials has stimulated interest in the development of biomarkers and other surrogate endpoints that may substitute for clinical endpoints, especially for the evaluation of treatments whose outcomes do not become evident for many years. The treatment of surrogate endpoints in the Medical and Statistics literature has often been heuristic and ad hoc in character. For instance, an inherent limitation of current surrogate endpoint validation techniques is its general failure in predicting outcome in treating diseases which are multifactorial in terms of the physiological and/or behavioral changes that may occur in populations suffering from the disease.

Statistical methods have been applied to find correlations between measured biochemical parameters and clinical outcomes. For example, U.S. Pat. Nos. 5,824,467 and 6,087,090 describe a statistical approach to the prediction of a patient's response to a drug based on a “biochemical profile”, in an effort to match a treatment regimen with patients for whom the regimen is likely to be suitable. U.S. Pat. Nos. 6,267,116 6,575,169, 6,578,582, 6,581,606 and 6,581,607 describe methods of mathematical analysis of surrogate marker measurements for dose adjustment during pharmacotherapy. U.S. Pat. No. 6,556,977 describes the application of neural networks to create an expert system for diagnosis of medical conditions, which employs non-linear prediction methods to analyze a collection of diagnostic input variables.

Early detection and diagnosis are important in the successful prevention and treatment of diabetic macular edema. Existing methods of detection and evaluation rely on the subjective evaluation of images obtained through photography and angiography. There have been efforts to replace such qualitative data with quantitative measurements. Macular thickness, for example, which is a measure of macular edema, is a quantitative measurement that has been found to correlate with visual acuity (Oshima et al., Br. J. Ophthalmol. 1999; 83:54-61), and has more recently been accepted as a surrogate endpoint in clinical trials

There is currently a need to develop more effective statistical techniques for identification of surrogate endpoints, for surrogate endpoint analysis, for using surrogate endpoints in clinical trials of experimental treatment regimens, and for monitoring the effectiveness of established treatment regimens in the practice of medicine. In particular, there is a need for methods for monitoring the effectiveness of therapeutic regimens that treat ocular diseases, especially where long-term improvements in visual acuity are a desired clinical outcome but are not readily detected in the short term, after initiation of the regimen.

### BRIEF DESCRIPTION OF THE INVENTION

The present invention relates to systems and methods for data acquisition and analysis of self-reported, behavioral, neurological, biochemical and/or physiological data in a manner which permits identification of surrogate endpoints, particularly in multifactorial diseases. The invention also provides for the use of such data and methods in monitoring the effectiveness of a treatment regimen.

The subject methods and systems can be used as part of a discovery program for new therapeutic candidates, for identification of unanticipated applications for drugs that were previously investigated in other therapeutic areas, as well as for monitoring the effectiveness of ongoing treatment of a disease with new or accepted treatment regimens. The methods of the invention are suitable for making other drug-related observations, including but not limited to:

interactions among over-the-counter (OTC) medicines;

interactions between prescription and OTC medicines;

interactions between any medicine and foods, beverages, nutraceuticals, vitamins, and mineral supplements;

interactions between certain drug groups and foods, beverages, medicines, etc.;

distinguishing characteristics among certain drug groups;

validating interactions which are based on very limited evidence but which may be of great interest (e.g., where a few users out of many thousands report a serious side effect from some combination of medicines and/or foods); and

identifying classes of patients who are likely to be at risk when using a particular medicine or combination.

The invention provides methods and apparatus for predicting the ability or effectiveness of a drug or combination of drugs to bring about a clinically relevant result. In general, the method is based on assessing the ability of a treatment regimen to achieve one or more surrogate endpoints predicted from multivariate analysis of behavioral, biochemical and/or physiological data. In particular, the subject methods and systems can be used to predict the clinical outcome for a program of treatment, such as part of a clinical or pre-clinical trial, or as part of a treatment regimen (i.e., to assess if a patient is responsive to a particular treatment, titrate dosages, etc.). The subject methods and systems can also be used in a drug discovery program, e.g., to identify compounds which are likely to be useful in treating a particular condition based on their ability to achieve one or more surrogate endpoints in a test animal system. The present invention also contemplates the use of the subject methods and systems to categorize drugs based on their surrogate endpoint “signatures”, and additionally contemplates that such signatures can be stored in databases for comparison with other drugs or test compounds. Still another contemplated use of the subject method is in the development or optimization of drug formulations, e.g., that require a particular biodistribution, release profile or other pharmacokinetic parameter.

The system of the present invention can also provide tools for visualizing trends in the dataset, e.g., for orienteering, to simplify user interface and recognition of significant correlations.

The invention also provides a pharmaceutical product for treatment of a disease, comprising a drug substance indicated for treatment of the disease and further comprising instructions for administration of the drug substance and for monitoring the effectiveness of the treatment regimen according to the method described above. Optionally, the indication of the drug substance for extended treatment may be conditioned on the results of the monitoring.

In a particular aspect, the invention provides methods for monitoring the effects of treatment of ocular diseases, such as macular degeneration, diabetic retinopathy, and the like, particularly those diseases associated with macular edema.

The present invention also contemplates methods of conducting informatics and drug discovery businesses utilizing the apparatus, methods and databases of the present invention.

### DETAILED DESCRIPTION OF THE INVENTION

1. Statistical Methods.

The invention provides a method for monitoring the effectiveness of a regimen for treatment of a disease. The method comprises obtaining, from one or more subjects, data in the form of measurements of one or more variables. Examples of suitable measurements include, but are not limited to, self-reported data (i.e., subjective or objective information reported by the patient) and behavioral, genetic, neurological, biochemical and physiological measurements. The same subject, or a different subject, is treated with the regimen for a selected period of time. The period of time may be any convenient period, ranging from hours to months or even years; it is selected by the practitioner and typically is based largely upon the expected rapidity of response to the regimen. From a subject who has been treated with the regimen, data in the form of measurements of one or more variables, as described above, are obtained, and changes in the measurements induced by the regimen are noted. For purposes of this operation, no observed change in a measurement is noted as a change having a value of zero.

The invention makes use of a “signature” which represents probability relationships between predictor variables and clinical outcomes (both favorable and unfavorable) for the disease being treated. Predictor variables include, but are not limited to, the values of measurements as described herein (before or after a treatment regimen), changes in such measurements induced by a treatment regimen, and mathematical combinations thereof as described further below.

The signature is derived from previous clinical outcomes and predictor variables derived from previous measurements and/or changes in measurements. The previous clinical outcomes do not need to have resulted from the treatment regimen being evaluated, but may have resulted from treatment with other regimens including but not limited to other drugs, therapies, and surgical procedures. For this purpose, spontaneous remission may also be regarded as a treatment regimen, because such remissions may be associated with a predictor variable. The identities of the predictor variables are determined by correlating previously-obtained clinical outcomes with previously-obtained measurements and/or mathematical combinations thereof, preferably by using at least one automated non-linear algorithm to detect and provide statistical probabilities for associations between the predictor variables and the outcomes. By comparing the signature to the experimental values of the predictor variables that are derived from measurements obtained from the subject treated with the regimen, it is possible to determine a probability that continued treatment of the subject with the regimen will eventually result in a favorable clinical outcome.

Alternatively, in certain embodiments of the invention, the predictor variables may be identified by correlating previously-obtained measurements (and/or mathematical combinations thereof) with pre-determined physiological states. These pre-determined physiological states will preferably be target states, reflecting a normal, healthy condition, or a state which is otherwise regarded as a target condition into which the subject is intended to be brought by the treatment regimen. For example, blood pressure within a normal range could be an element of a pre-determined physiological state, a state which a subject is not in when an antihypertensive treatment regimen is initiated. This need not be identical to a state corresponding to a favorable clinical outcome, which could involve an above-normal but nonetheless greatly improved blood pressure.

Predictor variables may be the values of measurements themselves. For example, the level of a particular tumor-specific antigen prior to treatment may be associated with a favorable or unfavorable outcome of a cancer treatment regimen. A predictor variable may also be related to changes in the measurements induced by a treatment regimen (e.g., a drop in viral load after initiation of a treatment regimen for AIDS). The measurements may optionally be converted to log values and/or normalized to some convenient range of numerical values.

Predictor variables may also be mathematical constructs obtained from linear or non-linear combinations of measurements and changes in measurements. For example, long-term survival of cancer patients treated with a given regimen might be weakly associated with changes in two or more independent measurements, while being more strongly correlated with the simultaneous presence of those changes in a single subject. A mathematical combination of the two or more measurements would then provide a predictor variable that correlates with the desirable clinical outcome more strongly than any of the individual measurements. The nature of such mathematical combinations are preferably determined empirically, so as to give the resulting predictor variables the highest degree of correlation with the clinical outcome.

Preferably, a large number of combinations such as sums, differences, products, ratios, and the like are examined between all possible pairings of measurements and derivatives thereof (roots, powers, logarithms, and the like), in each case evaluating the transformed data for association with clinical outcomes. Those combinations yielding higher “r” values may optionally be used in further combinations. Such pairings, mathematical combinations, and statistical evaluations are of course preferably carried out by a computer. The use of measurements and mathematical combinations thereof in this manner to arrive at predictive models for treatment regimens is described in more detail in U.S. Pat. Nos. 5,824,467 and 6,087,090, which are incorporated herein by reference in their entireties.

The identification and statistical weighting of associations between input variables and clinical outcomes may be done by any of the statistical methods accepted in the art. Methods employing non-linear algorithms represent preferred embodiments. The analysis and evaluation is preferably implemented on a computer system, and may employ a variety of statistical computation software packages that are known in the art. Artificial intelligence systems and other “expert system” designs are preferably employed, with artificial neural networks, particularly “fuzzy” neural networks, being especially preferred.

Essentially, the method of the invention seeks to identify a collection of markers and surrogate endpoints, or mathematical expressions derived therefrom, that are associated with favorable and unfavorable outcomes, and determines if the regimen being evaluated has a similar pattern of effects on those markers and surrogate endpoints. If a pattern of effects is observed which resembles the pattern associated with a favorable outcome, the treatment regimen is deemed likely to be effective, and treatment can be continued with some degree of confidence that a favorable clinical outcome will eventually result. Conversely, if the pattern of observed effects resembles the pattern associated with unfavorable outcomes, the treatment regimen is deemed likely to be ineffective or possibly haniful, depending on the outcomes associated with the observed pattern, and alternative treatment regimens can be substituted and similarly evaluated.

A salient feature of the subject method is that it can be used to establish surrogate endpoints for multifactorial disease. A surrogate endpoint is a laboratory measurement or a physical sign used as a substitute for a clinically meaningful endpoint that measures directly how a patient feels, functions or survives. Changes induced by a therapy on a surrogate endpoint are expected to reflect changes in a clinically meaningful endpoint. Many diseases involve multiple symptoms, the alleviation of which can, if definitively linked to the disease outcome, be used as a basis for selecting a drug candidate, obtaining regulatory (FDA) approval, and/or assessing and modifying treatment regimens for individual patients. Indeed, in many cases there is likely to be no one single surrogate endpoint will be appropriate because the disease is multifactorial, i.e., no on marker is predictive of the outcome of treatment.

The subject methods and systems address this problem by utilizing multi-dimensional analysis, such as classification techniques and/or association techniques, to establish a predictive relationship for disease treatment based on two or more independent factors which can be (readily) measured in the treated patients. Using combinations of machine learning, statistical analysis, modeling techniques and database technology, the subject method advantageously utilizes data mining techniques to find and identify patterns and relationships in patient data that permits inference of rules for the prediction of drug effects. Such surrogate endpoints can include, and be derived from analysis of biochemical, physiological and/or behavioral changes, including changes which manifest at the level of gross anatomical changes or as changes in cellular (gene expression or other phenotypic or genotypic changes) or metabolic profiles.

“Accuracy”, when applied to data, refers to the rate of correct values in the data. When applied to models, accuracy refers to the degree of fit between the model and the data. This measures how error-free the model's predictions are.

The term “API” refers to an application program interface. When a software system features an API, it provides a means by which programs written outside of the system can interface with the system to perform additional functions. For example, a data mining software system of the subject invention may have an API which permits user-written programs to perform such tasks as extract data, perform additional statistical analysis, create specialized charts, generate a model, or make a prediction from a model.

An “association algorithm” creates rules that describe how often behavioral, biochemical and/or physiological events have occurred together. Such relationships are typically expressed with a confidence interval.

The term “back propagation” refers to a training method used to calculate the weights in a neural net from the data.

The term “binning” refers to a data preparation activity that converts continuous data to discrete data by replacing a value from a continuous range with a bin identifier, where each bin represents a range of values. For example, changes in visual acuity could be converted to bins such as 0, 1-5, 6-10 and over 10.

The term “bioerodable polymer” refers to polymers which degrade in vivo, where erosion of the polymer over time is required to achieve sustained release of a pharmaceutical agent over time. Specifically, hydrogels such as methylcellulose which act to release drug through polymer swelling are specifically excluded from the term “bioerodable polymer”.

The terms “bioerodable” and “biodegradable” are equivalent and are used interchangeably herein.

“Categorical data” are labels or discrete categories into which the objects under study can be placed, based on one or more qualitative characteristics, as opposed to “measurement data” which is based on quantitative properties. Categorical data is either non-ordered (nominal), such as the gender or HIV status of a subject, or ordered (ordinal) such as high/low/no response to a stimulus.

The term “classification” refers to the problem of predicting the number of sets to which an item belongs by building a model based on some predictor variables. A “classification tree” is a decision tree that places categorical variables into classes.

A “clustering algorithm” finds groups of items that are similar. For example, clustering could be used to group physiological or biochemical markers according to statistical parameters of their predictive powers for certain biological consequences. It divides a data set so that records with similar content are in the same group, and groups are as different as possible from each other. When the categories are unspecified, this is sometimes referred to as unsupervised clustering. When the categories are specified a priori, this is sometimes referred to as supervised clustering.

The term “confidence” refers to a measure of how much more likely it is that B occurs when A has occurred. It is expressed as a percentage, with 100% meaning B always occurs if A has occurred. This can also be referred to this as the conditional probability of B given A. When used with association rules, the term confidence is observational rather than predictive.

“Continuous data” can have any value in an interval of real numbers. That is, the value does not have to be an integer. Continuous is the opposite of discrete or categorical.

“Controlled release” and “sustained release” are used interchangeably to refer to the release of a drug from a device or composition into surrounding tissue or physiological fluid at a predetermined rate. The rate of release can be zero order, pseudo-zero order, first order, pseudo-first order and the like, so long as relatively constant or predictably varying amounts of the drug can be delivered over an extended period of time, typically greater than 24 hours.

The term “degree of fit” refers to a measure of how closely the model fits the training data.

The term “discriminant analysis” refers to a statistical method based on maximum likelihood for determining boundaries that separate the data into categories.

The “dependent variables” (outputs or responses) of a model are the variables predicted by the equation or rules of the model using the independent variables (inputs or predictors).

The term “gradient descent” refers to a method to find the minimum of a function of many variables.

The “independent variables” (inputs or predictors) of a model are the variables used in the equation or rules of the model to predict the output (dependent) variable.

The term “itemset” refers to a set of items that occur together.

The phrase “k-nearest neighbor” refers to a classification method that classifies a point by calculating the distances between the point and points in the training data set. Then it assigns the point to the class that is most common among its k-nearest neighbors (where k is an integer).

The term “machine learning” refers to a computer algorithm used to extract useful information from a database by building probabilistic models in an automated way. “Measurement” as used herein refers to the obtaining of both measurement data and categorical data.

The term “mode” refers the most common value in a data set. If more than one value occurs the same number of times, the data is multi-modal.

A “model” can be descriptive or predictive. A “descriptive model” helps in understanding underlying processes or behavior. For example, an association model describes the effects of a drug on animal physiology as manifest in the measured behavior, physiology and/or biochemical markers. A “predictive model” is an equation or set of rules that makes it possible to predict an unseen or unmeasured value (the dependent variable or output) from other, known values (independent variables or input). For example, a predictive model can be used to predict side-effects of a drug in humans based on data for the drug when used in non-human animals.

A “node” is a decision point in a classification (i.e., decision) tree. Also, a point in a neural net that combines input from other nodes and produces an output through application of an activation function. A “leaf” is a node not further split—the terminal grouping—in a classification or decision tree.

An “ophthalmic disorder” refers to a physiologic abnormality of the eye. It may involve the retina, the vitreous humor, lens, cornea, sclera or other portions of the eye, or it may be a physiologic abnormality which adversely affects the eye, such as inadequate tear production or elevated intraocular pressure, or an imbalance in the concentration of a soluble species.

“Preventing vision degeneration” refers to preventing degeneration of vision in patients newly diagnosed as having a degenerative disease affecting vision, or at risk of developing a new degenerative disease affecting vision, and to preventing further degeneration of vision in patients who are already suffering from or have symptoms of a degenerative disease affecting vision.

“Promoting vision regeneration” refers to maintaining, improving, stimulating or accelerating recovery of, or revitalizing one or more components of the visual system in a manner which improves or enhances vision, either in the presence or absence of any ophthalmologic disorder, disease, or injury.

A “regression tree” is a decision tree that predicts values of continuous variables.

The term “significance” refers to a probability measure (p) of how strongly the data support a certain result (usually of a statistical test). If the significance of a result is said to be 0.05, it means that there is only a 5% probability that the result could have happened by chance alone. A very lowp value (p<0.05) is usually taken as evidence that the data mining model should be accepted since events with very low probability seldom occur. So if the estimate of a parameter in a model showed a significance of 0.01, that would be evidence that the parameter must be in the model.

“Supervised learning” refers to a data analysis using a well-defined (known) dependent variable. All regression and classification techniques are supervised. In contrast, “unsupervised learning” refers to the collection of techniques where groupings of the data are defined without the use of a dependent variable. The term “test data” refers to a data set independent of the training data set, used to evaluate the estimates of the model parameters (i.e., weights).

A “time series” is a series of measurements taken at consecutive points in time. Data mining methods of the present invention that handle time series can incorporate time-related operators such as moving average. “Windowing” is used when training a model with time series data. A “window” is the period of time used for each training case.

The term “time series model” refers to a model that forecasts future values of a time series based on past values. The model form and training of the model can take into consideration the correlation between values as a function of their separation in time.

The term “training data” refers to a data set independent of the test data set, used to fine-tune the estimates of the model parameters (i.e., weights).

Visual acuity is determined by asking a subject to read a Snellen eye chart from a distance of 20 feet. A subject who can resolve letters approximately one inch high at 20 feet is said to have 20/20 visual acuity, which is considered “normal” acuity. If the smallest letters a subject can resolve at 20 feet are letters that a person with 20/20 acuity can resolve at 40 feet, the subject is said to have “20/40 vision” or 20/40 acuity.

“Visualization” tools graphically display data to facilitate better understanding of its meaning. Graphical capabilities range from simple scatter plots to complex multi-dimensional and multi-colored representations.

2. Data Generation and Analysis

The patient data can include data pertaining to behavioral, neurological, genetic, biochemical and/or physiological activity or markers, as well as self-reported data provided by the patient. For instance, the data can include on one or more of sleeping, locomotion (including ambulatory and non-ambulatory movements, foot misplacement, and the like), body weight, anxiety, pain sensitivity, convulsions, intraocular pressure, cardiac response (e.g., output, QT interval), heart rate, blood pressure and body temperature, respiration (e.g., rate, O₂ or CO₂), circadian rhythms, visual acuity, physical measurements of body components (retinal thickness, tumor volume), learning, memory (short/long) and the like.

The subject methods can also utilize cellular and molecular marker data. For instance, changes in gene expression, levels of proteins, post-translational modification of proteins or other cellular structures (including extracellular markers), extracellular matrix composition or levels, tissue microarchitecture, metabolites, hormones or other natural small molecules, as well as the presence in the patient of genetic markers, such as particular phenotypes (e.g. antigen levels, protein isoforms), RFLPs, genotypes or haplotypes. Rates of cell growth, differentiation and/or death may also be useful in identifying certain surrogate endpoints.

By measuring a plurality of responses the methods of this invention provides a means for objectively finding surrogate markers which are predictive of the clinical endpoints that a treatment regimen is likely to induce in a patient. The methods also provide a means for objectively finding surrogate markers which are indicative of the clinical endpoint an ongoing treatment regimen is likely to achieve in a particular patient. The former process is one of prediction, based on previously collected data and applied to a patient prior to treatment, while the latter process is one of monitoring the progress of a treatment regimen based on contemporaneous data from a treated patient.

3. Database Analysis Techniques

Various data mining techniques can be used as part of the subject invention. In certain preferred embodiments, the data mining system uses classification techniques, such as clustering algorithms, which find rules that partition the database into finite, disjoint, and previously known (or unknown) classes. In other embodiments, the data mining system uses association techniques, e.g., of summarization algorithms, which find the set of most commonly occurring groupings of items. Yet in other embodiments, the data mining system uses overlapping classes.

In one embodiment, the subject method using a data mining technique based on association rules algorithms. These techniques derive a set of association rules of the form X [symbol] Y, where X and Y are sets of behavioral, neurological, biochemical and/or physiological responses and each drug administration is a set of literals. The data mining task for association rules can be broken into two steps. The first step consists of finding all large itemsets. The second step consists of forming implication rules with a user specified confidence among the large itemsets found in the first step. For example, from a dataset, one may find that an association rule such as drugs which slowed a decrease in visual acuity also cause a reduction in the rate of retinal thickening, or a decrease in intraocular pressure. Association rules can also be more complex, requiring that two or more criteria are met in order for the rule to evoked. A rule X [symbol] Y holds in the data set D with confidence c if c% of the occurrences of X in the data set also contain Y. The rule X [symbol] Y has support s in the data set if s% of the entries in D contain X [symbol] Y. Confidence is a measure of the strength of implication and support indicates the frequencies of occurring patterns in the rule.

Another technique that can be used in the methods of the present invention is the process of data classification. Classification is the process of finding common properties among a set of “objects” in a database, and grouping them into various classes based on a classification scheme. Classification models are first trained on a training data set which is representative of the real data set. The training data is used to evolve classification rules for each class such that they best capture the features and traits of each class. Rules evolved on the training data are applied to the main database and data is partitioned into classes based on the rules. Classification rules can be modified as new data is added.

Yet another data mining technique that can be used in the subject method is the use of sequential pattern mining. This technique can be used to find sequential patterns which occur a significant number of times in the database. This analysis can be used to detect temporal patterns, such as the manifestation of secondary adaptation or effects involving combinatorial therapies. Time-Series clustering is another data mining technique that can be used to detect similarities in different time series.

In yet another embodiment, the subject method uses a clustering method for finding correlations in the behavioral database(s). Clustering is the grouping together of similar data items into clusters. Clusters should reflect some mechanism at work in the domain from which instances or data points are drawn, a mechanism that causes some instances to bear a stronger resemblance to one another than they do to the remaining instances. If X is a set of data items, the goal of clustering is to partition X into K groups Cₖ such every data that belong to the same group are more “alike” than data in different groups. Each of the K groups is called a cluster. (G. Fung, Comprehensive Overview of Basic Clustering Algorithms, 2001; available at www.cs.wisc.edu/˜gfung/clustering.pdf). In general, clustering methods can be broadly classified into partitional and hierarchical methods.

Partitional clustering attempts to determine k partitions that optimize a certain criterion function. The square-error criterion is a good measure of the within-cluster variation across all the partitions. The objective is to find k partitions that minimize the square-error. Thus, square-error clustering tries to make the k clusters as compact and separated as possible, and works well when clusters are compact “clouds” of data points that are rather well separated from one another.

Hierarchical clustering is a sequence of partitions in which each partition is nested into the next partition in the sequence. An agglomerative method for hierarchical clustering starts with the disjoint set of clusters, which places each input data point in an individual cluster. Pairs of clusters are then successively merged until the number of clusters reduces to k. At each step, the pair of clusters merged are the ones between which the distance is the minimum. There are several measures used to determine distances between clusters. For example, pairs of clusters whose centroids or means are the closest are merged in a method using the mean as the distance measure (dₘₑₐₙ). This method is referred to as the centroid approach. In a method utilizing the minimum distance as the distance measure, the pair of clusters that are merged are the ones containing the closest pair of points (dₘᵢₙ). This method is referred to as the “all-points” approach.

In another embodiment, the subject method uses Principal Component Analysis (PCA). This is not a classification method per se. The purpose of PCA is to represent the variation in a data set into a more manageable form by recognizing classes or groups. The assumption in PCA is that the input is very high dimensional (tens to thousands of variables). PCA extracts a smaller number of variables that cover most of the variability in the input variables. As an example, suppose there are data along a line in 3-space. Normally one would use 3 variables to specify the coordinates of each data point. In fact, just 1 variable is needed: the position of the data point along the line that all the data lies on. PCA is a method for finding these reductions. An advantage to PCA is that it can be a reasonably efficient method whose reduction is well founded in terms of maximizing the amount of data variability explained while using a smaller number of variables.

Still another embodiment utilizes a neural net or neural network, e.g., a complex non-linear function with many parameters that maps inputs to outputs. Such algorithms may use gradient descent on the number of classification errors made, i.e. a routine is implemented such that the number of errors made decreases monotonically with the number of iterations. Gradient descent is used to adjust the parameters such that they classify better. An advantage to neural nets is that such algorithms can handle high dimensional, non-linear, noisy data well.

The neural net can be trained with “supervision”, i.e., a mechanism by which the net is given feedback by classifying its responses as “correct” or “incorrect”. It eventually homes into the correct output for each given input, at least with some probability. Such machine learning techniques may be advantageously employed for either or both of vision classification components or data mining components of the instant invention.

Supervised learning requires the buildup of a library of readily-classified data sets for input into the neural net. Although more economic in terms of the amount of data needed, supervised learning implies that only pre-determined classes can be ascribed to unseen data. To allow for the possibility of finding a novel therapeutic class, such as “antidepressant drugs with anti-manic component”, unsupervised clustering could be more appropriate.

In certain embodiments, a preferred method can combine both types of learning: a supervised learning of the neural net until it correctly classifies a basic training set, but which also utilizes unsupervised learning to further subdivide the trained classes into meaningful sub-classes or to add completely new sub-classes. The training and use of neural networks in predictive medicine, in the context of diagnosis, is described in more detail in U.S. Pat. No. 6,556,977, which is incorporated herein by reference in its entirety. Ando et al., Jpn. J Cancer Res. 2002; 93:1207-1212, have described the use of a fuzzy neural network in identifying correlations between gene expression profiles and prognosis in B-cell lymphoma. Schwarzer et al., Statistics in Medicine 2000, 19:541-561, provide a critical evaluation of the limitations of neural networks as applied to medical diagnosis and prognosis.

Principal component analysis (PCA) involves a mathematical procedure that transforms a number of (possibly) correlated variables into a (smaller) number of uncorrelated variables called principal components. The first principal component accounts for as much of the variability in the data as possible, and each successive component accounts for as much of the remaining variability as possible. Traditionally, principal component analysis is performed on a square symmetric matrix of type SSCP (pure sums of squares and cross products), Covariance (scaled sums of squares and cross products), or, Correlation (sums of squares and cross products from standardized data). The analysis results for matrices of type SSCP and Covariance do not differ. A correlation object is preferably used if the variances of individual variates differ much, or the units of measurement of the individual datapoints differ, such as is the case when the analysis comprises data from behavioral, neurological, biochemical and physiological measures. The result of a principal component analysis on such objects will be a new object of type PCA.

In still other embodiments, the subject method utilizes K-means and fuzzy clustering. Gaussian mixture models are a common version of this. These techniques are “unsupervised” clustering methods. They assume the user has no outputs, but would like to group the data anyway according to inputs that are similar to each other. The idea is to choose a model for each cluster. For example, each cluster may consist of points inside a hyper-sphere centered at some location in the input space. These methods automatically determine the number of clusters, place them in the correct places, and determine which points belong to which clusters. An advantage to these techniques is that they can be efficient algorithms and can do a good job of finding clusters. This is a method of choice when the user does not have a priori information about the classes

Another embodiment utilizes the hierarchical clustering Serial Linkage Method. This is an unsupervised clustering method in the same sense as K-means and fuzzy clustering. Here individual points are joined to each other by being close to each other in the input space. As these points are joined together, they define clusters. As the algorithm continues, the clusters are joined together to form larger clusters. Compared to K-means and fuzzy clustering, hierarchical clustering has the advantage that clusters can have arbitrary non-predefined shapes and the result correctly shows “clusters of clusters.” A disadvantage to these methods is they tend to be more sensitive to noise.

Yet another embodiment utilizes a nearest neighbor algorithm. This is a true supervised learning method. There is a set of training data (inputs, i.e. datapoints, and outputs, i.e. classes) that are given in advance and just stored. When a new query arrives, the training data is searched to find the single data point whose inputs are nearest to the query inputs. Then the output for that training data point is reported as the predicted output for the query. To reduce sensitivity to noise, it is common to use “k” nearest neighbors and take a vote from all their outputs in order to make the prediction.

In yet another embodiment, the subject method uses a logistic regression algorithm. This is related to linear regression (fitting a line to data), except that the output is a class rather than a continuous variable. An advantage is that is method provides a statistically principled approach that handles noise well.

Still another embodiment utilizes a Support Vector Machine algorithm. This also has a linear separator between classes, but explicitly searches for the linear separator that creates the most space between the classes. Such techniques work well in high dimensions. Yet another embodiment relies on a Bayes Classifier algorithm. The simplest form is a naive Bayes classifier. These algorithms build a probabilistic model of the data from each class. Unsupervised methods above may be used to do so. Then, based on a query, the model for each class is used to calculate the probability that that class would generate the query data. Based on those responses, the most likely class is chosen.

Yet another embodiment utilizes a Kohonen self organizing maps (SOM) clustering algorithm. These algorithms are related to neural nets in the sense that gradient descent is used to tune a large number of parameters. The advantages and disadvantages are similar to those of neural networks. In relation to neural networks, Kohonen SOM clustering algorithms can have the advantage that parameters can be more easily interpreted, though such algorithms may not scale up to high dimensions as well as neural nets can.

The subject databases can include extrinsically obtained data, such as known protein interactions of a drug, chemical structure, K_{d} values, Pₖ/P_{d} parameters, IC₅₀ values, ED₅₀ values, TD₅₀ values and the like.

4. Ocular Diseases and Macular Edema.

Ocular diseases include, among others, disorders of the retina and disorders of the uveal tract. Disorders of the retina include but are not limited to vascular retinopathies (e.g., arteriosclerotic retinopathy and hypertensive retinopathy), central and branch retinal artery occlusion, central and branch retinal vein occlusion, diabetic retinopathy (e.g., proliferative and non-proliferative retinopathies), age-related macular degeneration, senile macular degeneration, neovascular macular degeneration, retinal detachment, retinitis pigmentosa, retinal photic injury, retinal ischemia-induced eye injury, and various forms of glaucoma, such as primary glaucoma, chronic open-angle glaucoma, acute or chronic angle-closure glaucoma, congenital/infantile glaucoma, secondary glaucoma, and absolute glaucoma.

Other retinal disorders include edema and ischemic conditions. Macular and retinal edema are often associated with metabolic illnesses such as diabetes mellitus, and with cataract extraction and other surgical procedures upon the eye. Retinal ischemia can occur from either choroidal or retinal vascular diseases, such as central or branch retinal vein occlusion, collagen vascular diseases and thrombocytopenic purpura. Retinal vasculitis and occlusion is seen with Eales disease and systemic lupus erythematosus.

Disorders of the uveal tract include but are not limited to uveitis (anterior uveitis, intermediate uveitis, posterior uveitis, iritis, cyclitis, choroiditis), and inflammation associated with ankylosing spondylitis, juvenile rheumatoid arthritis, chronic iridocyclitis, Reiter's syndrome, pars planitis, toxoplasmosis, cytomegalovirus (CMV), acute retinal necrosis, toxocariasis, toxoplasmosis, birdshot choroidopathy, histoplasmosis (presumed ocular histoplasmosis syndrome), Behcet's syndrome, sympathetic ophthalmia, VogtKoyanagi-Harada syndrome, sarcoidosis, reticulum cell sarcoma, large cell lymphoma, syphilis, tuberculosis, endophthalmitis, and malignant melanoma of the choroids.

Uveitis refers to inflammation of the uveal tract. It includes iritis, cyclitis, iridocyclitis and choroiditis and usually occurs with inflammation of additional structures of the eye. These disorders have a variety of causes but are typically treated with systemic steroids, topical steroids, or cyclosporin.

Macular edema is a swelling (edema) in the macula, an area near the center of the retina of the eye. Macular edema is commonly associated with diabetic retinopathy, accelerated or malignant hypertension, uveitis, iritis, Eales disease, retinitis pigmentosa, and as a complication of other inflammatory syndromes. Local edema is also associated with multiple cytoid bodies as a result of AIDS. It is most commonly diagnosed by fluorescein or indocyanine green (ICG) angiography, a diagnostic test which uses a fundus camera to image the structures in the back of the eye. The degree of severity of macular edema can be directly measured using state-of-the-art instruments such as confocal infrared scanning laser tomography (SLT) or optical coherence tomography (OCT), as described in more detail below.

Methods of measuring the degree of macular edema include measuring the area, volume, or thickness (height or elevation) of the edema. Changes in the degree of macular edema may be determined by methods known in the art, such as fundus photography, fluorescein angiography, and the like, preferably by measurements of retinal thickness including but not limited to the use of confocal scanning laser ophthalmoscopes, optical coherence tomography scanners, and scanning retinal thickness analyzers. The severity of edema can be graded based on established standards, such as the International Clinical Classification of Diabetic Retinopathy, Severity of Diabetic Macular Edema, Detailed Table (Released by International Council of Ophthalmology in October 2002, and incorporated herein by reference). That scale has two major levels: Diabetic Macular Edema Absent, and Diabetic Macular Edema Present. In the latter case, it can be further divided into several levels of severity: mild, moderate, and severe Diabetic Macular Edema. The explanation of each can be found in the published standard. Databases of measurements from normal eyes are available, and such data can be used for comparison purposes.

Confocal scanning laser tomography (SLT) is a useful non-invasive diagnostic technique to quantitatively analyze macular disorders. It is especially useful for the primary assessment and follow-up studies of macular holes and central serous retinopathy.

SLT makes a quantitative measurement of a structure, such as the optic nerve, that can be viewed and assessed clinically without expensive equipment. This technology, in the form of the Heidelberg retina tomograph (HRT, Heidelberg Engineering GmbH), has been available for around 10 years. A compact version (the HRT II) has been released more recently for clinical use. The field of view is 15° and imaging can be performed through an undilated pupil. Images are monochromatic and the confocal optics enable the determination of a surface height map (topography). (Burk et al., Graefes Arch. Clin. Exp. Ophthalmol. 2000; 238:375-384).

An example of a commercial device for scanning laser polarimetry (SLP) is the GDx Access™ (Laser Diagnostic Technologies, Inc., San Diego, Calif.). In this device, a polarized laser scans the fundus, building a monochromatic image. The state of polarization of the light is changed (retardation) as it passes through birefringent tissue, in this case the cornea and retinal nerve fiber layer (RNFL). After anterior segment compensation, which corrects for the birefringence of the cornea, the polarization retardation in light reflected from the fundus is converted into a measure of RNFL thickness. Although a change in RNFL thickness due solely to edema may not manifest itself as a change in retardance (M. Banks et al., Arch. Ophthalmol. 2003; 121:484-490), SLP measurements (with and without anterior segment compensation) can be taken and used as inputs in the method of the invention. Any association of these variables with clinical outcomes will be detected and assigned an appropriate level of significance.

Optical coherence tomography (OCT) is a noncontact, noninvasive imaging technique used to obtain high resolution (approximately 10 μm) cross-sectional images of the retina. OCT is analogous to ultrasound B-scan imaging except that light rather than sound waves are used. The device performs a linear scan on the retina with a near infrared, low coherence light beam. OCT software locates borders (changes in reflectivity) such as the vitreoretinal interface, the interface between RNFL and inner retinal layers, and the outer retina/choroid interface. OCT has been shown to be clinically useful for imaging selected macular diseases including macular holes, macular edema, age-related macular degeneration, central serous chorioretinopathy, epiretinal membranes, schisis cavities associated with optic disc pits, and retinal inflammatory diseases. In addition, OCT has the capability of measuring RNFL thickness in glaucoma and other diseases of the optic nerve. The dimensions of any of the various imaged structures may be used to generate input variables in the method of the present invention.

Laser optical cross-sectioning can be carried out using a commercial instrument called a retinal thickness analyzer (RTA), available from Talia Technology Ltd., Neve Ilan, Israel. The RTA projects a narrow slit of green laser light at an angle on the retina and acquires an image from a different angle on a digital camera. An optical cross-section of the retina is seen, with reflectance peaks that correspond to the RNFL/inner limiting membrane and the retinal pigment epithelium. The distance between the peaks is measured and processed by software to obtain retinal thickness, and optic disc topography can be carried out. The macula, peripapillary area and optic disc may be scanned.

Fundus photographs can be taken of the patients' eye in order to determine their macular edema assessments. An assessment may be converted to a numerical score, such as for example the “ETDRS level”, either through visual examination and scoring of 2-D fundus photographs, or with the aid of a digital camera and a 3-dimensional imaging system (S. Fransen et al., Opthalmology 2002; 109:595-601). A stereoscopic optic disc camera, such as the Discam™ available from Marcher Enterprises Ltd., or the DR-3DT digital camera system, available from Inoveon Corp, Oklahoma City, Okla., may be employed for 3-D imaging of the optic disc and macula. The devices provide a high-magnification, stable, stereoscopic picture that can be easier to evaluate than the image obtained with indirect ophthalmoscopy. Software enables the observer to make magnification-corrected measurements of optic disc features.

The topographic mapping and measurement techniques described above are useful for longitudinally monitoring patients for the development of macular edema, for monitoring patients during treatment, and for following the resolution of edema after treatment. In addition to generating quantitative data for use in the statistical methods of the invention, these imaging techniques can provide false-color maps of retinal thickness provide an intuitive and efficient method of comparing retinal thickness over several visits, which could be directly compared with slit-lamp observation.

5. Products and Methods of the Invention.

In a specific embodiment of the invention, the treatment regimen will comprise administration of one or more drugs that may affect visual acuity. In this particular embodiment, the disease may be, for example, a macular disease. Macular diseases include but are not limited to macular holes, macular edema, age-related macular degeneration, central serous chorioretinopathy, epiretinal membranes, schisis cavities, and retinal inflammatory diseases. The invention also provides pharmaceutical products which include one or more pharmaceutical formulations indicated for treatment of an ocular disease, and instructions for assessing a patient to whom the pharmaceutical formulation is administered and who presents some degree of macular edema and/or thickening of the retinal nerve fiber layer (RNFL). In one embodiment, the instructions direct the measurement of macular or retinal edema or RNFL thickening, which may involve measuring the area, volume, and/or thickness (height or elevation) of the edema and/or RNFL. In one embodiment, the instructions direct monitoring the degree of macular edema in the patient for about 2-18 months, preferably 6-12 months.

In certain of these embodiments, the instructions will direct altering the dosage regimen if the degree of macular edema does not decrease after administration of said formulation. In other embodiments, the instructions will direct terminating administration of the formulation in favor of another treatment regimen. For example, the instructions may specify that a certain minimum degree of clearance of edema is predictive of a reduced probability that the patient will experience a greater than or equal to a 15-letter loss in visual acuity within one year, and that a measured clearance of edema that meets or exceeds this minimum degree of clearance indicates that a positive clinical outcome is probable and that treatment with the regimen should therefore continue.

In one particular embodiment, changes in a measurement (retinal thickness) that are regarded as being associated with a clinical outcome (a long-term changes in visual acuity) are used to monitor a treatment regimen for macular edema, and to inform treatment decisions. The assessment of severity of edema may be accomplished by comparing a diseased edematous macula with a normal macula, followed by grading the severity of edema. Such grading scores, and/or measured parameters of the edema, may be used to derive variables for the method of the invention.

Pharmaceutical compositions useful in the invention include formulations intended for tiopical, oral or parenteral administration. Parenteral administration may involve systemic administration, for example intramuscular or intravenous injection, or may involve local injection, including but not limited to intraocular injection, subretinal injection, subscleral injection, intrachoroidal injection, and subconjunctival injection.

In specific embodiments, the pharmaceutical formulation is a sustained-released formulation, which may be provided in the form of a sustained-release device. Examples of such embodiments include but are not limited to sustained-release ocular products marketed under the tradenames RETAANE™, VITRASERT™, ENVISION TD™ and POSURDEX™.

In additional embodiments, the formulation may be delivered using a device employing sustained-release technologies sold under the tradenames AEON™ or CODRUG™.

In certain embodiments, the ophthalmic disorder is: posterior uveitis, Diabetic Macular Edema (DME), Wet Age-Related Macular Degeneration (ARMD), or CMV retinitis. In certain embodiments, the pharmaceutical formulation comprises one or more of an anti-inflammatory agent such as a corticosteroid or NSAID, an antiviral agent, an antibiotic agent a neuroprotective agent, an angiostatic agent such as anecortave, and/or an immunomodulatory agent such as cyclosporin A, FK506, and the like.

In specific embodiments, the pharmaceutical formulation includes an anti-inflammatory corticosteroid. Examples of suitable anti-inflammatory corticosteroids include, but are not limited to, acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, and triamcinolone hexacetonide. In a preferred embodiment, the steroidal antiinflammatory agent is selected from the group consisting of cortisone, dexamethasone, hydrocortisone, methylprednisolone, prednisolone, prednisone, and triamcinolone, and derivatives thereof such as acetonides and lower alkanoate esters such as acetates, propionates, and butyrates. Particularly preferred corticosteroids are triamcinolone acetonide (TA) and fluocinolone acetonide (FA).

The above lists of drugs are not meant to be exhaustive. Practically any approved or experimental drug may be used in the instant invention, and there are no particular restrictions in terms of molecular weight, solubility, or other physical properties.

In certain embodiments, the sustained-release formulation or device is capable of releasing active ingredients the over a period of about 1 month to about 20 years, preferably over a period of about 6 months to about 5 years. In one embodiment, the sustained release device is an intraocular implant, i.e., an implantable controlled-release drug delivery device, sized for implantation within an eye, and configured for continuous delivery of the pharmaceutical formulation within the eye for a period of at least several weeks. Such devices typically comprise a polymeric outer layer that is substantially impermeable to the drug contained therein, covering a core comprising a pharmaceutical formulation, where the outer layer has one or more orifices that create a flow path through which fluids may pass to contact the core and through which dissolved drug may pass to the exterior of the device.

In certain embodiments, the device further includes one or more semi-permeable layers disposed in the flow path, which semi-permeable layers are at least partially permeable to dissolved drug, wherein said semi-permeable layers reduce influx of proteins from ocular fluid and/or reduce the rate of release of dissolved drug from the device. In one embodiment, the rate of release of drug is determined solely by the composition of the core and the total surface area of the one or more orifices relative to the total surface area of said device. The outer layer may comprise polytetrafluoroethylene, polyfluorinated ethylenepropylene, polylactic acid, polyglycolic acid, or silicone or a mixture thereof.

In one embodiment, the outer layer is biodegradable. In one embodiment, the semipermeable layer comprises PVA. In certain embodiments, the drug or drugs comprise about 50-80 weight percent of the implant. Suitable sustained-release devices and compositions include but are not limited to those described in U.S. Pat. Nos. 5,378,475, 5,476,511, 5,773,019, 5,824,072, 5,902,598, 6,217,895, 6,375,972, 6,416,777, and 6,548,078. It should be understood that all embodiments described above may be combined with one another whenever appropriate and advantageous.

Another aspect of the invention provides a method for assessing the long term effect on visual acuity (VA) of a pharmaceutical formulation for treatment in a patient who presents some degree of macular edema, the method comprising assessing degree of macular edema before and after said treatment, wherein a reduction in said severity is predictive of increased long term benefit of improvement in visual acuity, and/or decreased long term risk of deterioration in visual acuity. The treatment may be directed to a condition unrelated to an ophthalmic disorder, and the effect may be a side-effect of the treatment.

Another aspect of the invention provides a method for conducting a drug discovery business, comprising:

(i) obtaining, from a test animal or from stored data, one or more measurements selected from the group consisting of behavioral, neurological, biochemical and physiological measurements;

(ii) treating said test animal with a test compound for a selected period of time;

(iii) obtaining, from a test animal treated with the regimen, one or more measurements selected from the group consisting of behavioral, neurological, biochemical and physiological measurements;

(iv) determining changes in the measurements induced by the regimen, by comparing the measurements obtained in (i) with the measurements obtained in (iii);

(v) comparing said measurements or changes in the measurements, or both, to a signature, said signature representing probability relationships between one or more predictor variables and one or more clinical outcomes for said disease; and

(vi) determining, from the comparison data of step (ii), the suitability of further clinical development of the test compound.

The identities of the predictor variables are determined by correlating pre-determined physiological states, or responses to known drugs, with previously-obtained measurements. Such measurements include but are not limited to: self-reported data and behavioral, genetic, neurological, biochemical and physiological measurements, and mathematical combinations thereof. The correlations are preferably derived by using at least one automated non-linear algorithm.

The above method may, in certain embodiments, also include conducting therapeutic profiling of test compounds determined to be suitable for further clinical development. Such profiling will typically include testing for efficacy and toxicity in animals.

The method may, in certain further embodiments, also include the preparation of structural analogues of a test compound determined to be suitable for further clinical development, and it may include conducting therapeutic profiling of the analogues. Structural analogues of test compounds are chemical compounds having substantially the same chemical structure as the test compound, but varying in the identity and/or position of chemical substituents. Examples include, but are not limited to, structures having one or more substitutions and/or relocations on the parent structure of hydrogen atoms, halogen atoms, lower alkyl groups, lower alkoxy groups, and other substituents, one for another, as well as derivatives of functional groups, such as esters of hydroxyl or carboxyl groups, amides of amino groups or carboxyl groups, and so forth. Structural analogs may also feature replacement of a ring structure in the parent test compound with a different ring structure of similar size, such as for example substitution of a benzene ring with a thiophene or pyridine ring, or vice-versa. The conception and preparation of structural analogues is a well-established process, well known to those of skill in the art of medicinal chemistry.

In further embodiments, the method may further include the licensing of a test compound determined to be suitable for further clinical development, or a structural analog thereof, to another business for clinical trials in human subjects. The method may also include licensing such a compound to a manufacturer, for manufacture and sale of a pharmaceutical preparation comprising the compound.

Another aspect of the invention provides a method of marketing a treatment for an ophthalmic disorder, comprising: (A) marketing, to healthcare providers, a pharmaceutical formulation for long-term treatment of said ophthalmic disorder, which formulation includes one or more drug substances that may affect visual acuity when administered over a sustained period of time; and, (B) providing to said healthcare providers instructions for administering said formulation, which instructions include a direction to assess a patient's prognosis with respect to long-term visual acuity by monitoring the effectiveness of treatment with the drug substance by measuring changes, if any, of macular edema as a prediction of visual acuity.

In one embodiment, the disease is a macular disease, and the drug substance is one that is indicated for the treatment of macular disease.

The invention also provides a method of marketing a treatment of an ocular disease or other ophthalmic disorder, comprising marketing to healthcare providers a drug substance indicated for treatment of an ophthalmic disorder (e.g. macular disease), and providing to the to healthcare providers instructions for monitoring the effectiveness of a treatment regimen as described above, where the regimen comprises administration of the indicated drug substance.

Another aspect of the invention provides a product for treatment for an ophthalmic disorder, comprising a pharmaceutical formulation for long-term treatment of said ophthalmic disorder, which formulation includes one or more drug substances that may affect visual acuity when administered over a sustained period of time; and instructions for administering said formulation, which instructions include a direction to assess a patient's prognosis with respect to long-term visual acuity by monitoring the effectiveness of treatment with the drug substance by measuring changes, if any, of macular edema as a prediction of visual acuity.

In one embodiment, the disease is a macular disease, and the drug substance is one that is indicated for the treatment of macular disease.

The invention also provides a pharmaceutical product for treatment of an ocular disease or other ophthalmic disorder, comprising a drug substance indicated for treatment of an ophthalmic disorder (e.g. macular disease), and instructions for monitoring the effectiveness of a treatment regimen as described above, where the regimen comprises administration of the indicated drug substance.

The product, comprising both drug substance and instructions, may be provided in a single package, or the instructions may be provided separately in a human-readable or computer-readable format. In certain embodiments, a database containing information about the associations between measurements and clinical outcomes, and the significance of those associations, is also provided a component of the product. Provision of the database may be effected by providing it on human-readable or computer-readable media; provision may also be effected by providing the purchaser with remote access to a database held on a computer or server.

### EXAMPLE

Edema is caused by a build-up of fluid in the retina that can affect the photoreceptor nerve cells lining the back of the eye, resulting in impaired vision. A phase III randomized, controlled and masked clinical trial study was conducted to assess the safety and efficacy of a fluocinolone acetonide implant for the treatment of diabetic macular edema (DME). The study was designed and powered to demonstrate a difference in the resolution of edema between patients treated with a fluocinolone acetonide implant and those treated with the standard of care. In this multi-center trial, 80 patients were randomized to receive standard of care (macular grid laser or observation) or either a 0.5 mg or a 2 mg fluocinolone acetonide implant. This implant, distributed under the trade name RETISERT™, is a small drug reservoir implanted into the back of the eye that delivers sustained and consistent levels of the drug fluocinolone acetonide directly to the affected area of the eye for up to three years. Enrollment of patients for the 2 mg dose was discontinued early in the trial due to side effects.

The primary endpoint for the study was a resolution in macular edema, as evidenced by a score of zero for retinal thickness at the center of the macula. At the 12-month follow-up, 48.8% of the patients treated with the 0.5 mg implant had a reduction of their retinal thickness scores to zero (resolution of macular edema), compared to 25.0% of those receiving standard of care (p<0.05). This is an almost 100% improvement over the standard of care.

Although the study was not designed or powered to demonstrate improvement in visual acuity and other secondary endpoints, these measures were evaluated and differences assessed between patients treated with the 0.5 mg implant and those treated with standard of care. At 12 months, patients treated with the 0.5 mg implant were more likely to show improvement in visual acuity of 15 letters or more compared to patients treated with the standard of care (19.5% vs. 7.1%). Also, implant-treated patients were less likely to have a decrease of 15 or more letters of visual acuity than were those in the standard of care group (4.9% versus 14.3%). Although the data did not reach statistical significance, possibly due to sample size limitation, the trends are encouraging. Over 70% of patients treated with the 0.5 mg implant had improved or stable visual acuity, compared to 50% of those treated with standard of care (p=0.08). Finally, more patients in the standard of care group had a worsening of their diabetic retinopathy score at twelve months (29.6%) compared to those receiving the 0.5 mg implant (5.1%).

These unexpected data indicate that there is a correlation between a short-term reduction in retinal thickness measurements (an indicator of macular edema) with an increased long-term improvement in visual acuity, and/or a decreased long-term risk of deterioration in visual acuity. Thus, a treatment regimen for DME, with a long-term endpoint of improved visual acuity (or reduced risk of loss of acuity), may be monitored in the short term by measurements of retinal thickness, with those measurements serving as predictors of the long-term outcome. A decision to continue or discontinue the regimen may be informed by the results of the short-term measurements.

Those skilled in the art will recognize, or be able to ascertain using no more than routine experimentation, many equivalents to the specific embodiments of the invention described herein. Such equivalents are intended to be encompassed by the following claims.

I claim:

1. A method for monitoring the effectiveness of a regimen for treatment of a disease, comprising:
(i) obtaining, from a subject, one or more measurements selected from the group consisting of self-reported data and behavioral, genetic, neurological, biochemical and physiological measurements;
(ii) treating said subject, or a different subject, with said regimen for a selected period of time;
(iii) obtaining from a subject who has been treated with the regimen, one or more measurements selected from the group consisting of self-reported data and behavioral, genetic, neurological, biochemical and physiological measurements;
(iv) determining changes in the measurements induced by the regimen, by comparing the measurements obtained in (i) with the measurements obtained in (iii);
(v) comparing said measurements or changes in the measurements, or both, to a signature, said signature representing probability relationships between one or more predictor variables and one or more clinical outcomes for said disease; and
(vi) determining, from the comparison in step (v), a probability that continued treatment of the subject with the regimen will result in a favorable clinical outcome;
wherein the identities of the predictor variables are determined by correlating previously-obtained clinical outcomes with previously-obtained measurements selected from the group consisting of self-reported data and behavioral, genetic, neurological, biochemical and physiological measurements, and mathematical combinations thereof, said correlations being derived by using at least one automated non-linear algorithm.

2. The method of claim 1, wherein the disease is ocular disease, the clinical outcome is an increase in visual acuity, and the measurement is a measure of retinal thickness.

3. The method of claim 2, wherein the disease is macular disease.

4. The method of claim 2, wherein the measure of retinal thickness is obtained by a means selected from the group consisting of confocal scanning laser ophthalmoscopes, optical coherence tomography scanners, and scanning retinal thickness analyzers.

5. The method of claim 3, wherein the measure of retinal thickness is obtained by a means selected from the group consisting of confocal scanning laser ophthalmoscopes, optical coherence tomography scanners, and scanning retinal thickness analyzers.

6. The method of claim 2, wherein the treatment regimen comprises administration of an anti-inflammatory corticosteroid.

7. The method of claim 3, wherein the treatment regimen comprises administration of an anti-inflammatory corticosteroid.

8. The method of claim 4, wherein the treatment regimen comprises administration of an anti-inflammatory corticosteroid.

9. The method of claim 5, wherein the treatment regimen comprises administration of an anti-inflammatory corticosteroid.

10. The method of claim 6, wherein the anti-inflammatory corticosteroid is administered via an intraocular implant.

11. The method of claim 7, wherein the anti-inflammatory corticosteroid is administered via an intraocular implant.

12. The method of claim 8, wherein the anti-inflammatory corticosteroid is administered via an intraocular implant.

13. The method of claim 9, wherein the anti-inflammatory corticosteroid is administered via an intraocular implant.

14. The method of claim 10, wherein the corticosteroid is fluocinolone acetonide or triamcinolone acetonide.

15. The method of claim 11, wherein the corticosteroid is fluocinolone acetonide or triamcinolone acetonide.

16. The method of claim 12, wherein the corticosteroid is fluocinolone acetonide or triamcinolone acetonide.

17. The method of claim 13, wherein the corticosteroid is fluocinolone acetonide or triamcinolone acetonide.

18. A pharmaceutical product for treatment of an ocular disease, comprising:
(i) a drug substance indicated for treatment of a macular disease; and
(ii) instructions for monitoring the effectiveness of a treatment regimen according to the method of any one of claims 2-17;
wherein the treatment regimen comprises administration of the indicated drug substance.

19. A pharmaceutical product according to claim 18 wherein the drug substance and the instructions are packaged together.

20. A pharmaceutical product according to claim 18, further comprising means for accessing a database containing one or more signatures representing probability relationships between changes measurements selected from the group consisting of self-reported data, behavioral, neurological, biochemical, or physiological responses, and clinical outcomes for macular disease.

21. A pharmaceutical product according to claim 19, further comprising means for accessing a database containing one or more signatures representing probability relationships between changes measurements selected from the group consisting of self-reported data, behavioral, neurological, biochemical, or physiological responses, and clinical outcomes for macular disease.

22. A pharmaceutical product according to claim 18, wherein at least one of the measurements is a measurement of retinal thickness.

23. A pharmaceutical product according to claim 19, wherein at least one of the measurements is a measurement of retinal thickness.

24. A pharmaceutical product according to claim 20, wherein at least one of the measurements is a measurement of retinal thickness.

25. A pharmaceutical product according to claim 21, wherein at least one of the measurements is a measurement of retinal thickness.

26. A pharmaceutical product according to claim 22, wherein the clinical outcome is an improvement in visual acuity.

26. A pharmaceutical product according to claim 23, wherein the clinical outcome is an 15 improvement in visual acuity.

26. A pharmaceutical product according to claim 24, wherein the clinical outcome is an improvement in visual acuity.

26. A pharmaceutical product according to claim 25, wherein the clinical outcome is an improvement in visual acuity.

27. A method for treating an ocular disease, comprising administering a drug indicated for treatment of an ocular disease, and monitoring the effectiveness of said administration by the method of any of claims 2-17.

28. A method for conducting a drug discovery business, comprising:
(i) obtaining, from a test animal or from stored data, one or more measurements selected from the group consisting of behavioral, neurological, biochemical and physiological measurements;
(ii) treating said test animal with a test compound for a selected period of time;
(iii) obtaining, from a test animal treated with the regimen, one or more measurements selected from the group consisting of behavioral, neurological, biochemical and physiological measurements;
(iv) determining changes in the measurements induced by the regimen, by comparing the measurements obtained in (i) with the measurements obtained in (iii);
(v) comparing said measurements or changes in the measurements, or both, to a signature, said signature representing probability relationships between one or more predictor variables and one or more clinical outcomes for said disease; and
(vi) determining, from the comparison data of step (ii), the suitability of further clinical development of the test compound;
wherein the identities of the predictor variables are determined by correlating pre-determined physiological states, or responses to known drugs, with previously-obtained measurements selected from the group consisting of self-reported data and behavioral, genetic, neurological, biochemical and physiological measurements, and mathematical combinations thereof; said correlations being derived by using at least one automated non-linear algorithm.

29. The method of claim 28, further comprising conducting therapeutic profiling of a test compound determined to be suitable for further clinical development for efficacy and toxicity in animals.

30. The method of claim 28, further comprising preparing a structural analogue of a test compound determined to be suitable for further clinical development, and conducting therapeutic profiling of said analogue for efficacy and toxicity in animals.

31. The method of claim 29 or claim 30, further comprising licensing a test compound determined to be suitable for further clinical development, or an analog thereof, to another business for clinical trials in human subjects.
